(19) United States
(12) Patent Application Publication
(10) Pub. No.: US 2014/0024946 A1
(43) Pub. Date: Jan. 23, 2014

(54) Apparatus, System and Method For Objectifying and Accessing Skin Conditions

(71) Applicant: Neal Schwartz, Ellicott City, MD (US)

(72) Inventor: Neal Schwartz, Ellicott City, MD (US)

(21) Appl. No.: 13/550,650

(22) Filed: Jul. 17, 2012

(51) Int. Cl. A61B 6/00

(52) U.S. Cl. 600/476

(57) ABSTRACT

A new method of rapidly objectifying and accessing an acne situation using numerical data and a scale measuring the severity of a particular skin condition and charted over time to allow easy identification of progress of the treatment therefor. The apparatus or software program which is also copyright protected, can be used to both numerically diagnose severity of a skin condition and to chart progress. The software apparatus can also be embeddable to other health related sites as an application to use on their site and a marketing tool. The application can be applied to anything visible on the skin such as bumps, dry patches, dark marks, cuts, scabs, scars or anything visible and useful for counting and objectifying the number or severity of the affliction.

### BACKGROUND OF THE INVENTION

1. Field of the Present Invention

The present invention relates generally to a new apparatus, system and method for rapidly objectifying and accessing skin condition. More particularly the invention includes an apparatus, system and method using numerical data and a novel numerical based system for evaluating severity and treatment success of various skin conditions.

2. Description of the Related Art

Various skin conditions are difficult to treat and track the success or failure of such treatment. For example, in the treatment of acne, the treatment periods can be measured in weeks and months. As such a patient, and more importantly, a health care provider, needs a quick and easy way to assess a treatment strategy with readily apparent data.

Typically, photos of the affected area are taken and compared over specified time frames. However this method does not allow for immediate assessment of a treatment strategy. The photos must be compared every time a new photo is taken. Photos often times cannot be readily utilized as objective data because the process of healing, dark marks, and scars can obscure the overall aesthetic image and confuse both patient and practitioner in regards to the presence or absence of objective progress. This new method eliminates the need for an artistic eye and instead creates objective numerical data that is irrefutable. This objective data is very reassuring to a patient who doubts their treatment choices based on the aesthetic distractions mentioned above.

The addition of computer programs have helped with the assessment of such treatments in that photos can be compared online. However the current state of the art does not allow for immediate objective assessment of a condition or effectiveness of a particular treatment.

Therefore, there exists a need for an apparatus, system or method objectifying and accessing skin conditions and treatments.

### SUMMARY OF THE INVENTION

In a first aspect the present invention relates generally to a new apparatus, system and method for rapidly objectifying and accessing an acne situation. More particularly the invention includes an apparatus, system and method using numerical data and a novel scale system for evaluating acne severity and treatment success.

The method includes the steps of collecting multiple photographs of an affected skin condition over a pre-determined time period, identifying specific skin conditions in the affected area for each collected photograph, charting progress of the identified skin conditions for severity and treatment based upon a pre-determined scale, where the predetermined scale measures the skin condition and treatment thereof using a pre-determine numerical data quantitatively assessing the skin conditions in the affected area for each collected photograph, and charting progress of the identified skin conditions in a easy to visualize graph of trajectory over time. This rapid quantitative study clinically represents improvement or lack of improvement in the type of skin conditions that accumulate like acne, warts growths, scabs, dry patches, etc.

In some embodiments the predetermined scale includes a scaling where “each mark=1.” A trajectory of progress vs. a lack of progress is the concept in the preferred embodiment where zero is the goal regardless of the scaling system starting number. The preferred embodiment tracks progress of a tangible number of marks. The preferred embodiment can track progress, the lack of progress, trajectory, a starting situation and accumulation where zero bumps and marks equals the ultimate outcome.

In embodiments where each mark=1, marks can be added up automatically as they are marked by the apparatus giving a final objective number that can be graphed for trajectory. This provides a comprehensive objectified finding to an otherwise confusing aesthetic analysis. The preferred embodiment turns a subjective assessment into an irrefutable objective assessment that can be used as tangible evidence to show progress or lack thereof.

The apparatus includes a computer system having a self contained memory, a graphical user interface and at least one peripheral device connected thereto, the self contained memory having computer software code, where the software is programmed with instructions to collect multiple photographs of an affected skin condition over a pre-determined time period, identify specific skin conditions in the affected area for each collected photograph, chart progress of the identified skin conditions for tracking progress and treatment based upon a pre-determined scale, where the predetermined scale measures the skin condition and treatment thereof using a pre-determine numerical data.

The computer system further includes computer software programmed with instructions to allow uploading of a uses photos to an online database that is designed to store such photos in a predetermined catalogue manner. The computer system further includes software programmed with instructions to identify and individually mark specific skin conditions in the uploaded photographs. The computer system further includes computer software programmed with instructions to chart progress of the marked skin conditions and the treatment thereof. The individual marking includes the ability to color code and specifically select a size and shape of the mark for simple referencing and charting.

As used herein pre-determined time periods include the relative progress over time in general of a particular skin condition ant its treatment. Relative change can be objectified day to day or on a linear graph.

Also as used herein the word “treatment” can include “Progression of treatment” or “effectiveness of treatment”. Each patient progresses day to day and their progress can be tracked, over a predetermined amount of time for the treatment. What is novel is proving relative improvement over time.

### BRIEF DESCRIPTION OF THE DRAWINGS

While the specification concludes with claims particularly pointing out and distinctly claiming the present invention, it is believed the same will be better understood from the following description taken in conjunction with the accompanying drawings, which illustrate, in a non-limiting fashion, the best mode presently contemplated for carrying out the present invention, and in which like reference numerals designate like parts throughout the Figures, wherein:

FIG. 1 is a block diagram generally illustrating an exemplary distributed computing system with which an embodiment of the present invention can be implemented;

FIG. 2 is a flow diagram showing a series of steps which can be implemented according to one embodiment of the present invention;

FIG. 3 is a flow diagram showing an additional series of steps which can be implemented according to one embodiment of the present invention;

FIG. 4 is a screen shot of a user's personal website showing acne analytics according one embodiment of the invention; and

FIG. 5 is a screen shot of a user's personal website showing a before photo of an affected skin area with identified and marked areas thereon according to one embodiment of the invention.

### DETAILED DESCRIPTION OF THE PREFERRED EMBODIMENTS

The present disclosure will now be described more fully with reference to the Figures in which an embodiment of the present disclosure is shown. The subject matter of this disclosure may, however, be embodied in many different forms and should not be construed as being limited to the embodiments set forth herein.

A new method of rapidly objectifying and accessing an acne situation using numerical data and a scale measuring the severity of a particular skin condition and charted over time to allow easy identification of progress of the treatment therefor.

The apparatus or software program which is also copyright protected, can be used to both numerically diagnose severity of a skin condition and to chart progress.

The software apparatus can also be embeddable to other health related sites as an application to use on their site and a marketing tool

The application can be applied to anything visible on the skin such as bumps, dry patches, dark marks, cuts, scabs, scars or anything visible and useful for counting and objectifying the number or severity of the affliction.

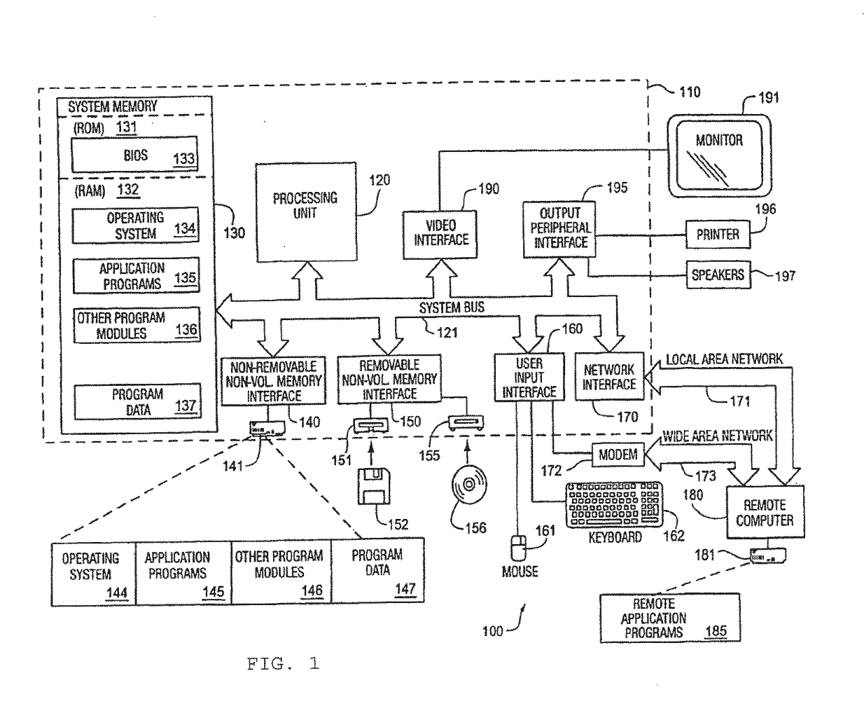

FIG. 1 and the following discussion are intended to provide a brief general description of a suitable computing environment 100 in which an example embodiment of the invention may be implemented. As used herein, the terms “computing system,” “computer system,” and “computer” refer to any machine, system or device that comprises a processor capable of executing or otherwise processing program code and/or data. Examples of computing systems include, without any intended limitation, personal computers (PCs), minicomputers, mainframe computers, thin clients, network PCs, servers, workstations, laptop computers, hand-held computers, programmable consumer electronics, multimedia consoles, game consoles, satellite receivers, set-top boxes, automated teller machines, arcade games, mobile telephones, personal digital assistants (PDAs) and any other processor-based system or machine. The term “data” refers to any information of any form, including commands, transfers, notifications, or requests. The terms “program code” and “code” refer to any set of instructions that are executed or otherwise processed by a processor.

The term “tree data storage structure” refers to any storage system or structure such as binary trees, B-trees or variants of B-trees, or other similar structures that provide an indexing of stored information. The tree data storage structure includes nodes branching out from a root node, where each node may include information consisting of one or more keys, an operation (such as less-than or greater-than) and/or information, typically referred to as a “pointer,” regarding a descendent node. Such structures may be used for storing large files of information and such information may be stored on a secondary storage device such as a disk. The term “identifier” refers to a form of or a substitute for a pointer of a node of a tree data storage structure that may be used to identify only one past or present node of a tree data storage structure and is used to map the node to a location in persistent storage. The term “cache” refers to a high-speed storage mechanism that may be either a reserved section of main memory or an independent high-speed storage device.

The term “operation” refers to functions performed using a tree data storage structure such as, for example, reading or writing to a node of the structure. The term “lock” refers to a function used in multi-computer or network systems to maintain file or information integrity or consistency by preventing conflicting operations. That is, if an operation is being performed, then no conflicting operations will be allowed. A lock acquisition may be required before each operation to ensure that operations do not conflict.

While a general purpose computer is described below, this is merely one example. The present invention also may be operable on a thin client having network server interoperability and interaction. Thus, an example embodiment of the invention may be implemented in an environment of networked hosted services in which very little or minimal client resources are implicated, e.g., a networked environment in which the client device serves merely as a browser or interface to the World Wide Web.

Although not required, the invention can be implemented via an application programming interface (API), for use by a developer or tester, and/or included within the network browsing software which will be described in the general context of computer-executable instructions, such as program modules, being executed by one or more computers (e.g., client workstations, servers, or other devices). Generally, program modules include routines, programs, objects, components, data structures and the like that perform particular tasks or implement particular abstract data types. Typically, the functionality of the program modules may be combined or distributed as desired in various embodiments. An embodiment of the invention may also be practiced in distributed computing environments where tasks are performed by remote processing devices that are linked through a communications network or other data transmission medium. In a distributed computing environment, program modules may be located in both local and remote computer storage media including memory storage devices.

FIG. 1 illustrates an example of a suitable computing system environment 100 in which the invention may be implemented, although as made clear above, the computing system environment 100 is only one example of a suitable computing environment and is not intended to suggest any limitation as to the scope of use or functionality of the invention. Nor should the computing environment 100 be interpreted as having any dependency or requirement relating to any one or combination of components illustrated in the exemplary operating environment 100.

With reference to FIG. 1, an example system for implementing the invention includes a general purpose computing device in the form of a computer 110. Components of computer 110 may include, but are not limited to, a central processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus (also known as Mezzanine bus).

Computer 110 typically includes a variety of computer readable media. Computer readable media can be any available media that can be accessed by computer 110 and includes both volatile and nonvolatile, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, random access memory (RAM), read-only memory (ROM), Electrically-Erasable Programmable Read-Only Memory (EEPROM), flash memory or other memory technology, compact disc read-only memory (CDROM), digital versatile disks (DVD) or other optical disk storage, magnetic cassettes, magnetic tape, magnetic disk storage or other magnetic storage devices, or any other medium which can be used to store the desired information and which can be accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of any of the above are also included within the scope of computer readable media.

The system memory 130 includes computer storage media in the form of volatile and/or nonvolatile memory such as ROM 131 and RAM 132. A basic input/output system 133 (BIOS) containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137. RAM 132 may contain other data and/or program modules.

The computer 110 may also include other removable/non-removable, volatile/nonvolatile computer storage media. By way of example only, FIG. 1 illustrates a hard disk drive 141 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156, such as a CD ROM or other optical media. Other removable/non-removable, volatile/nonvolatile computer storage media that can be used in the example operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1 provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 110 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus 121, but may be connected by other interface and bus structures, such as a parallel port, game port or a universal serial bus (USB).

A monitor 191 or other type of display device is also connected to the system bus 121 via an interface, such as a video interface 190. In addition to monitor 191, computers may also include other peripheral output devices such as speakers 197 and printer 196, which may be connected through an output peripheral interface 195.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

A computer 110 or other client device can be deployed as part of a computer network. In this regard, the present invention pertains to any computer system having any number of memory or storage units, and any number of applications and processes occurring across any number of storage units or volumes. An embodiment of the present invention may apply to an environment with server computers and client computers deployed in a network environment, having remote or local storage. The present invention may also apply to a standalone computing device, having programming language functionality, interpretation and execution capabilities.

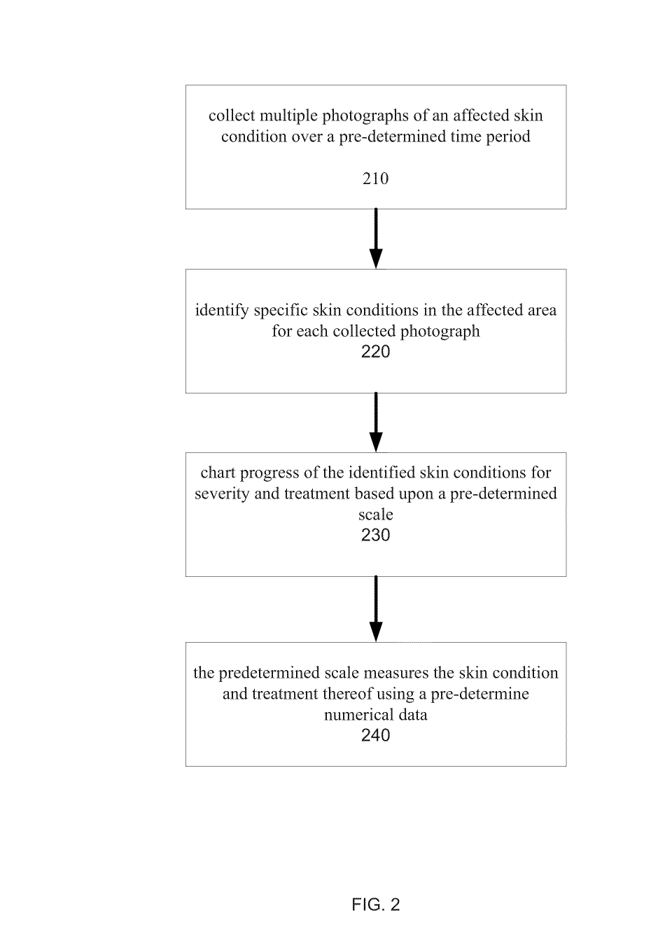

Referring now to FIG. 2, there is shown a series of steps in connection with the method according to one embodiment of the present invention. The steps in FIG. 2 can be instructions of a computer software program in connection with the computer system according to one embodiment of the present invention. In box 210 the system and method collects multiple photographs of an affected skin condition over a pre-determined time period. Exemplary time periods can be in hours, days, weeks or months. In box 220 the system and method indentifies specific skin conditions in the affected area for each collected photograph. Such conditions can be areas of an inflammatory condition, discoloration, bumps, rough patches, dry skin, razor bumps or folliculitis. In box 230 the system and method charts progress of the identified skin conditions for severity and treatment based upon a pre-determined scale. The preferred embodiment includes charting forward or backward progress of the identified skin conditions using a numerical assessment of the predetermined-scale as a clinical guide. The pre-determined scale may include a numerical and linear scale where 100 is much worse than 5. Zero would be the goal and represents “total inactivation”, e.g. zero inflammation and zero risk of further scarring.

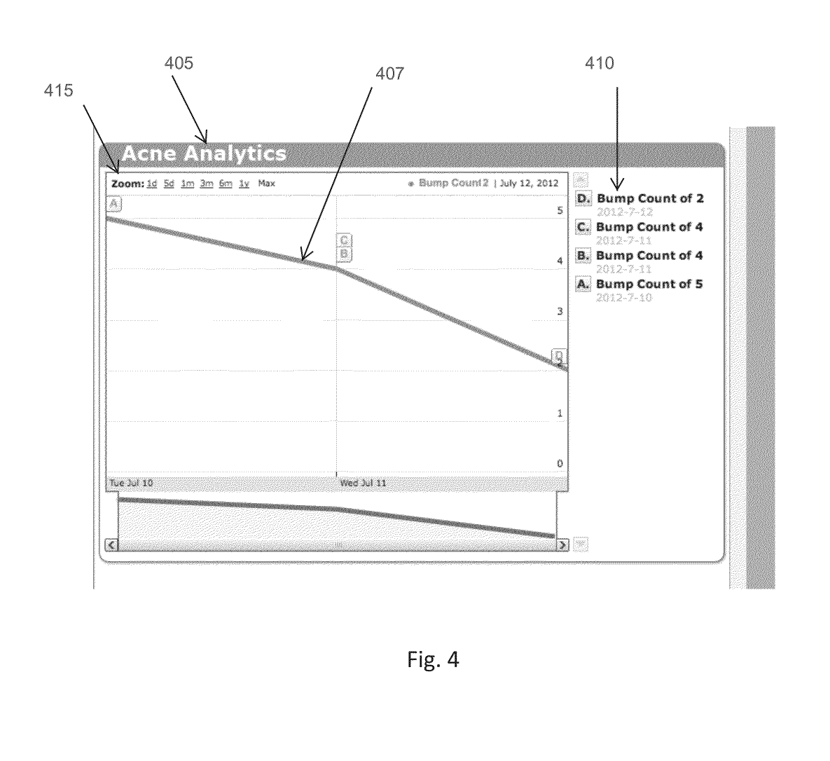

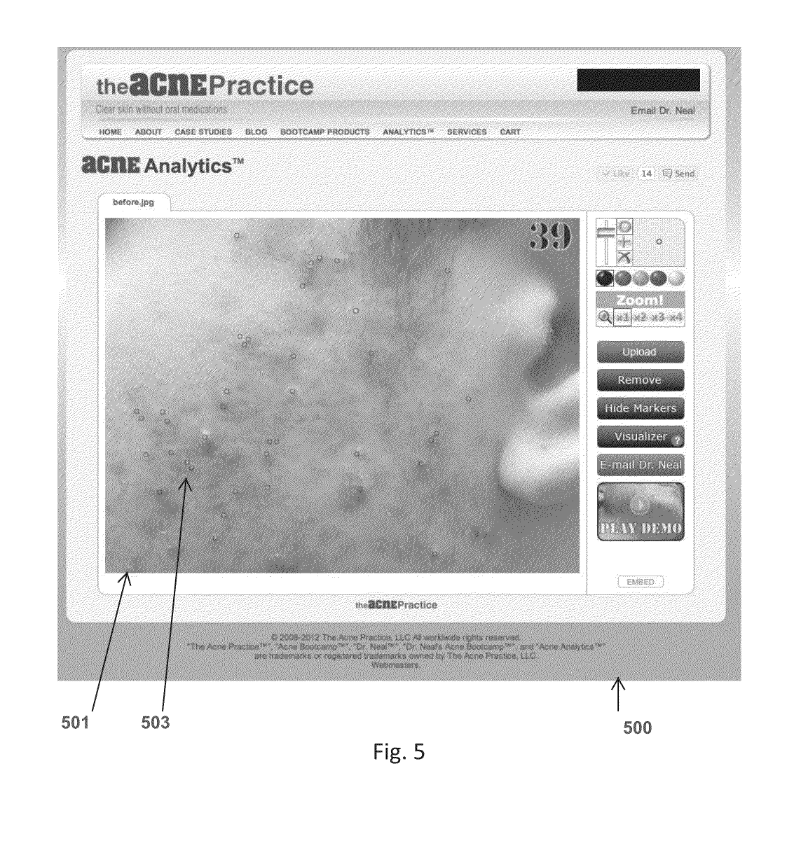

In box 240 the system and method includes a predetermined scale which measures the skin condition and treatment thereof using a pre-determined numerical data. All data is totaled per session and then graphed to show a trajectory of clinical improvement or worsening over time. See FIGS. 4 and 5 below for additional description of the scale.

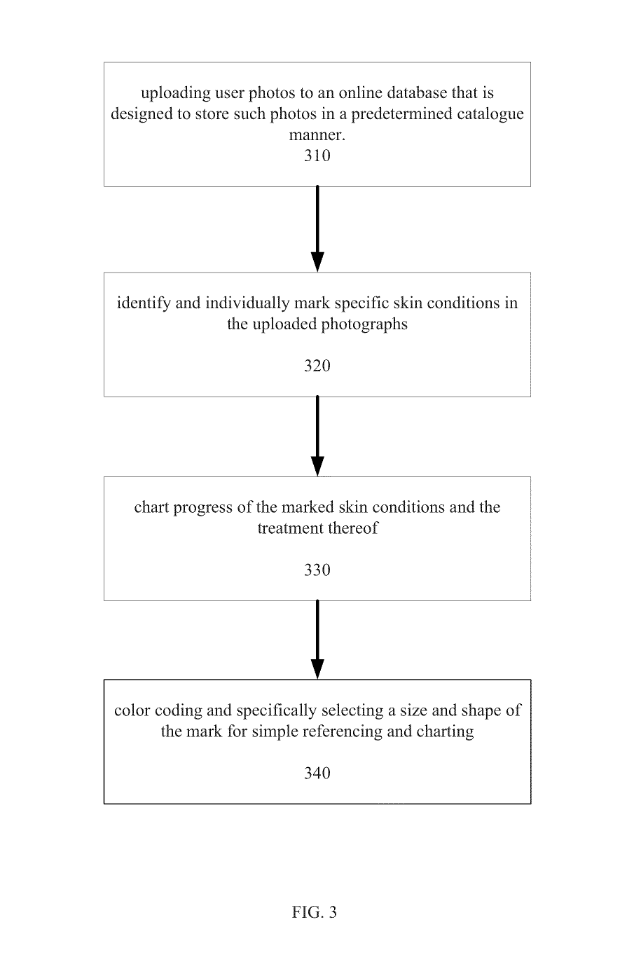

Referring now to FIG. 3 the system and method further includes additional steps according to one embodiment of the present invention. The steps in FIG. 3 can be instructions of a computer software program in connection with the computer system according to one embodiment of the present invention. The computer software instructions can be programmed to allow uploading of a uses photos to an online database that is designed to store such photos in a predetermined catalogue manner (box 310). In box 320 the system and method further includes software instructions to identify and individually mark specific skin conditions in the uploaded photographs. In box 330 the system and method further includes computer software instructions to chart progress of the marked skin conditions and the treatment thereof. In box 340 the system and method further includes individual skin condition marking providing the ability to color code and specifically select a size and shape of the mark for simple referencing and charting.

Referring now to FIG. 4 there is shown a screen shot a user's acne analytics report 405 showing day to day bump counts 410 for a given affected area. The scale 407 in this embodiment shows a decrease in bumps over a single day period; however additional period scaling 415 can be achieved for five day, one month, three month, six month and one year periods. Additional periods may also be achievable.

Referring now to FIG. 5 there is shown a user's personal website 500 with users account profile activated and a first picture of affect area 501 showing a before picture of affected skin area with identified and marked bump clusters 503. The system according to the present invention preferably compares at least two photographs taken over a series of time periods to show differences in identified affect skin areas.

It will be apparent to one of skill in the art that described herein a novel apparatus, system or method objectifying and accessing skin conditions and treatments. While the invention has been described with reference to specific preferred embodiments, it is not limited to these embodiments. The invention may be modified or varied in many ways and such modifications and variations as would be obvious to one of skill in the art are within the scope and spirit of the invention and are included within the scope of the following claims.

1. A method for objectifying a skin condition, the method include the steps of:
collecting multiple photographs of an affected skin condition over a pre-determined time period,
identifying specific skin conditions in the affected area for each collected photograph, and
charting progress of the identified skin conditions for severity and treatment based upon a pre-determined scale, where the predetermined scale measures the skin condition and treatment thereof using a pre-determine numerical data.

2. The method according to claim 1 further including the step of uploading of a user's photos to an online database that is designed to store such photos in a predetermined catalogue manner.

3. The method according to claim 2 further including identifying and marking specific skin conditions in the uploaded photographs.

4. The method according to claim 3 further including the step of tracking progress of the marked skin conditions and the treatment thereof.

5. The method according to claim 4 further including the step of individually marking each of the skin conditions.

6. The method according to claim 5 further including the step of color coding and specifically selecting a size and shape of the marked skin condition.

7. A computer system having a self contained memory, a graphical user interface and at least one peripheral device connected thereto, the self contained memory having computer software code with instructions to collect multiple photographs of an affected skin condition over a pre-determined time period, identify specific skin conditions in the affected area for each collected photograph, chart progress of the identified skin conditions for severity and treatment based upon a pre-determined scale, where the predetermined scale measures the skin condition and treatment thereof using a pre-determine numerical data.

8. The computer system according to claim 7 further including computer software instructions to allow uploading of a uses photos to an online database that is designed to store such photos in a predetermined catalogue manner.

9. The computer system according to claim 8 further including computer software instructions to identify and individually mark specific skin conditions in the uploaded photographs.

10. The computer system according to claim 9 further including computer software instructions to chart progress of the marked skin conditions and the treatment thereof.

11. The computer system according to claim 10 further including individually marking each of the skin conditions.

12. The computer system according to claim 11 further including color coding and specifically selecting a size and shape of the marked skin condition.
